(19) United States
(12) Patent Application Publication
(10) Pub. No.: US 2024/0342291 A1
(43) Pub. Date: Oct. 17, 2024

(54) Therapeutic Coordination Polymers Containing Pharmaceuticals for Drug Release Applications

(71) Applicant: UNIVERSITY OF WINDSOR, Windsor (CA)

(72) Inventors: Vedran VUKOTIC, LaSalle (CA); Jennifer Nicole MURPHY, Guelph (CA); Michelle DAO, Windsor (CA); Joy-Lynn KOBTI, Windsor (CA)

(21) Appl. No.: 18/681,718

(22) PCT Filed: Aug. 5, 2022
(86) PCT No.: PCT/CA2022/000035

§ 371 Date: Feb. 6, 2024

Related U.S. Application Data
(60) Provisional application No. 63/230,289, filed on Aug. 6, 2021.

(51) Int. Cl. A61K 47/52; A61K 47/54

(52) U.S. Cl. CPC A61K 47/52; A61K 47/545

(57) ABSTRACT

In a preferred embodiment, there is provided a pharmaceutical coordination composition having a plurality of central atoms or ions, a multitopic ligand and one or more pharmaceutical compounds coordinated to at least one of the plurality of the central atoms or ions, wherein the multitopic ligand is coordinated to at least two of the plurality of central atoms or ions.

### FIELD OF THE INVENTION

This invention relates to a pharmaceutical coordination complex, macrocycle or polymer having a central atom or ion, a pharmaceutical compound coordinated to the central atom or ion and at least one multitopic ligand, and which preferably includes one or more said multitopic ligand coordinated to two or more of said central atom or ion.

### BACKGROUND OF THE INVENTION

The ability to encapsulate drug molecules and control the rate of their release from a host material may permit the development of optimal drug release systems. Whether drugs are administered orally, through injection, or via a post-surgical implant, optimizing their release rate is relevant to maximizing their therapeutic effect. By utilizing a host material, the drug release rate may be altered. Previously, such materials including biodegradable organic polymers impregnated with drug molecules have been studied. Such systems may, however, suffer from low drug loadings and suboptimal control of their drug release rate.

Many pharmaceuticals suffer from poor solubility and non-ideal drug release kinetics that could be improved by an appropriate drug release material. Solubility, controlled release, and uptake of pharmaceuticals have indeed presented challenges in providing a vehicle or formulation to deliver pharmaceuticals and achieve the intended effect with improved patient compliance.

### SUMMARY OF THE INVENTION

A possible non-limiting object of the present invention is to provide a pharmaceutical coordination composition having a pharmaceutical compound and a ligand coordinated to a central atom or ion, and which may permit for improved controlled release of the compound and reduced side effects.

The applicant has appreciated that inorganic or coordination polymers may permit controlled release of a drug with increased drug loadings and improved drug release over hours, days, weeks or months. The coordination composition or polymer may also permit increased drug bioavailability, extended drug release and improved drug absorption. The coordination composition may also be formed with biocompatible components to reduce an immunogenic response.

In one aspect, the present invention provides a pharmaceutical coordination composition having a plurality of central atoms or ions, a multitopic ligand and one or more pharmaceutical compounds coordinated to at least one of the plurality of the central atoms or ions, wherein the multitopic ligand is coordinated to at least two of the plurality of central atoms or ions.

In one embodiment, the coordination composition comprises a coordination polymer or macrocycle. In one embodiment, the macrocycle comprises two to twelve said central atoms or ions. In one embodiment, the coordination polymer is a branched or unbranched linear coordination polymer. In an alternative embodiment, the coordination polymer is a nonlinear coordination polymer, whereby at least one said central atom or ion is coupled to more than two other said central atoms or ions with the multitopic ligand interposed therebetween. In one embodiment, the nonlinear coordination polymer includes the central atoms or ions arranged in a two-dimensional structure.

In one embodiment, the coordination composition is a coordination polymer, each said central atom or ion having a coordination number between 3 and 8, inclusive, and at least one said central atom or ion is coordinated with one to six said pharmaceutical compounds. In one embodiment, the coordination number is between 3 and 6, inclusive, and at least one said central atom or ion is coordinated with one to three said pharmaceutical compounds and/or two said multitopic ligands, and zero to three solvent molecules. In one embodiment, at least one said central atom or ion is coordinated with two or four said pharmaceutical compounds. In one embodiment, the multitopic ligand is a ditopic ligand which makes a single coordinate bond to two different central atoms or ions. In one embodiment, the multitopic ligand is also a bidentate ligand which makes two coordinate bonds to each said central atoms or ions. In one embodiment, each said central atom or ion in the coordination polymer has a geometry selected from the group consisting of trigonal planar, tetrahedral, square planar, trigonal bipyramidal, square pyramidal, octahedral, trigonal prismatic, pentagonal bipyramidal, capped octahedral, capped trigonal prismatic, square antiprismatic, dodecahedral, bicapped trigonal prismatic, cubic and hexagonal bipyramidal.

It is to be appreciated that the central atom or ion may have a solvent molecule coordinated thereto, such as water or alcohol, such as but not limited to methanol, ethanol, propanol, butanol or pentanol.

In one embodiment, the central atoms or ions comprise metal or transition metal atoms or ions. It is to be appreciated that the central atoms or ions are not strictly limited, provided that the central atom is operable to form the coordination composition or coordinate with the multitopic ligand. In one embodiment, the central atom or ion comprises a metal or transition metal having an oxidation state between 0 and 7, inclusive. In one embodiment, the central atom or ion comprises a lanthanide or actinide element, preferably Gd, Gd¹⁺, Gd²⁺ or Gd³⁺. In one embodiment, the metal or transition metal is a metal or transition metal ion having an oxidation state of +1, +2 or +3, or preferably, +2. In one embodiment, the central atoms or ions comprises one or more of Zn, Cu, Mn, Mg, Fe, Ca and Ag, or one or more of Zn²⁺, Cu²⁺, Mn²⁺, Mg²⁺, Fe²⁺, Fe³⁺, Ca²⁺ and Ag¹⁺. In one embodiment, the central atoms or ions comprises one or more of Zn, Cu, Mn or Mg.

In one embodiment, the metal is selected from the group consisting of Mg²⁺ and Ca²⁺. In one embodiment, the transition metal is selected from the group consisting of Ag¹⁺, Ru²⁺, Ru³⁺, Cr²⁺, Cr³⁺, Rh³⁺, Rh²⁺, Al, Si, Mn²⁺, Fe²⁺, Fe³⁺, Co²⁺, Co³⁺, Ni²⁺, Cu¹⁺, Cu²⁺ and Zn²⁺, or preferably, the transition metal is one or more of Ag¹⁺, Cr²⁺, Cr³⁺, Mn²⁺, Fe²⁺, Fe³⁺, Co²⁺, Co³⁺, Cu¹⁺, Cu²⁺ and Zn²⁺. In one embodiment, the transition metal is one or more of Ag¹⁺, Mn²⁺, Cu¹⁺, Cu²⁺ and Zn²⁺, or the transition metal is one or more of Mn²⁺ and Zn²⁺.

It is to be appreciated that the multitopic ligand (otherwise referred to as a linker) is not particularly limited, provided that the ligand is operable to form coordinate covalent bonds with two or more different central atoms or ions, such as a Lewis base. In one embodiment, the multitopic ligand comprises a plurality of coordinating portions and a linking portion interposed between the coordinating portions, wherein the plurality of the coordinating portions comprises at least first and second coordinating portions each respectively selected to form first and second coordination bonds with one and another one of the plurality of the central atoms or ions. By way of non-limiting examples, the coordinating portions may include hydroxyl, halo, alkyl, alkenyl, alkynyl, cycloalkyl, heterocycloalkyl, aryl, aralkyl, heteroaryl, heteroaralkyl, formyl, haloformyl, carbonyl, carboxyl, alkoxy, alkoxycarbonyl, (alkoxycarbonyl)oxy, carbamoyl, amino, imino, imido, azo, azido, cyanato, isocyanato, nitroxy, cyano, isocyano, nitro, sulfanyl, alkylsulfanyl, sulfinyl, sulfino, thiocyanate, isothiocyanato or a combination thereof.

In one embodiment, one or both of the first and second coordinating portions comprise optionally substituted heterocycloalkyl or heteroaryl. In one embodiment, said heterocycloalkyl or heteroaryl comprises five- or six-membered heterocycloalkyl or heteroaryl optionally having one or more heteroatoms each selected from the group consisting of a nitrogen atom, an oxygen atom, a sulfur atom and a phosphorus atom. In one embodiment, the heteroatom is a nitrogen atom. In one embodiment, said heterocycloalkyl or heteroaryl is selected from the group consisting of imidazolyl, methylimidazolyl, ethylimidazolyl, 1H-1,2,3-triazolyl, 4H-1,2,4-triazolyl, 1H-tetrazolyl, nicotinate, and isonicotinate. In any of these embodiments, any hydrogen atom could be substituted with a group that does not substantially adversely affect the stability or activity of the compound. In one embodiment, said imidazolyl is 2-methylimidazolyl or 2-ethylimidazolyl. In one embodiment, said heterocycloalkyl or heteroaryl is one or more of the following (with the stippled lines indicating connection to the linking portion):

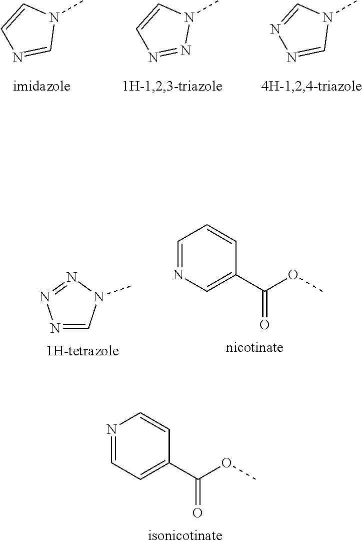

In another embodiment, one or both of the first and second coordinating portions comprise a ligand which contains a primary alkyl amine or secondary amine, such as glycinamide which can coordinate to the metal center in a monodentate or bidentate fashion via nitrogen and or oxygen donors as a chelating ligand (stippled lines indicating connection to the linking portion):

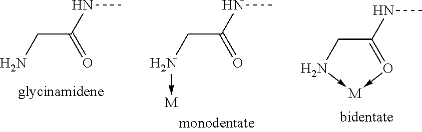

Likewise, the linking portion is not particularly limited, provided the linking portion can be coupled to the coordinating portions, and may be selected depending on various desired physical, mechanical, chemical, or pharmaceutical properties of the coordination composition, such as drug release rate. In one embodiment, the linking portion comprises optionally substituted branched or unbranched C₁-C₃₀ alkylene, alkenylene or alkynylene,

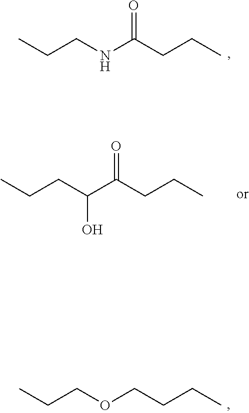

wherein one or more carbon atoms of said C₁-C₃₀ alkylene, alkenylene or alkynylene is optionally substituted with a nitrogen atom, an oxygen atom or a sulfur atom. In one embodiment, said C₁-C₃₀ alkylene includes methylene, ethylene, n-propylene, isopropylene, butylene, pentylene, hexylene or octylene. In one embodiment, the linking portion comprises:

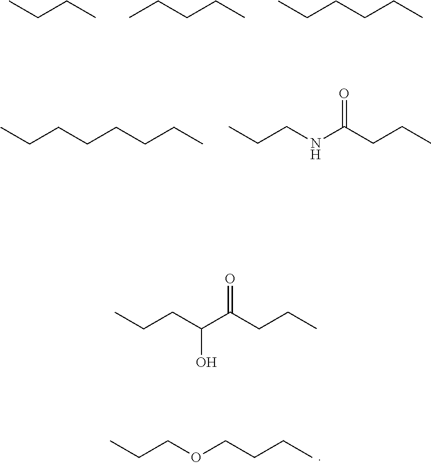

In one embodiment, the multitopic ligand comprises two said coordinating portions coupled to opposing ends of the linking portion. In one embodiment, the linking portion is a branched linking portion having one or more branching portions. In one embodiment, the multitopic ligand comprises 1,1′-(1,4-butanediyl)bis(imidazole); 1,1′-(1,5-pentanedidyl) bis(imidazole); 1,1′-(1,6-hexanedidyl) bis(imidazole); 1,1′-(1,8-octanedidyl) bis(imidazole); or

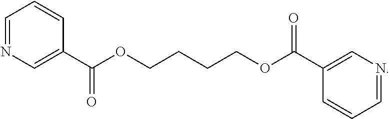 [SMILES: O=C(OCCCCOC(=O)c1cccnc1)c1cccnc1]

In one embodiment, the multitopic ligand comprises one or more of the following:

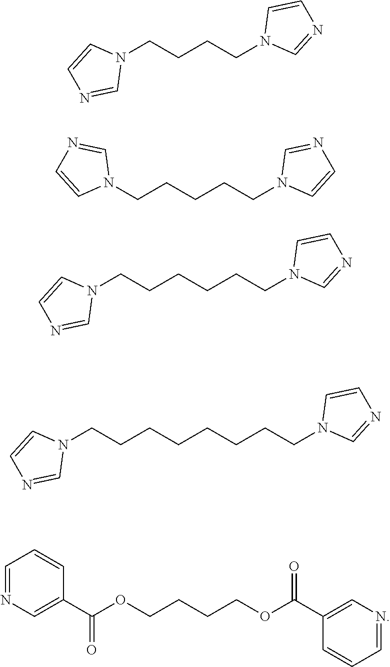

In one embodiment, the multitopic ligand comprises one or more of the following:

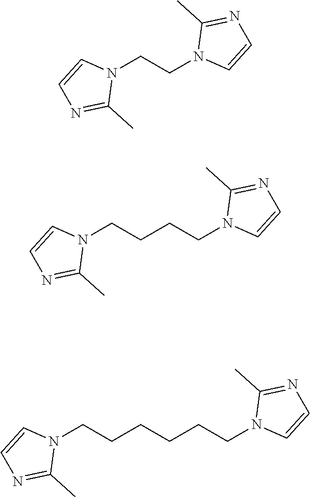 [SMILES: Cc1nccn1CCn1ccnc1C ; Cc1nccn1CCCCn1ccnc1C ; Cc1nccn1CCCCCCn1ccnc1C]

In one embodiment, the multitopic ligand comprises one or more of the following:

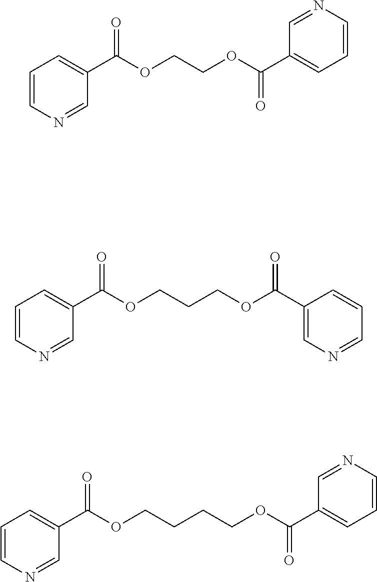 [SMILES: O=C(OCCOC(=O)c1cccnc1)c1cccnc1 ; O=C(OCCCOC(=O)c1cccnc1)c1cccnc1 ; O=C(OCCCCOC(=O)c1cccnc1)c1cccnc1]

It has been appreciated that the above multitopic ligand with a pair of nicotinate coordinating portions may include the alkylene linking portion with more than four carbon atoms. In one embodiment, the linking portion comprises butylene, pentylene, hexylene or octylene.

In one embodiment, the multitopic ligand comprises one of the following:

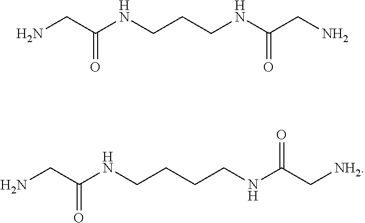 [SMILES: NCC(=O)NCCCNC(=O)CN ; NCC(=O)NCCCCNC(=O)CN]

In one embodiment, the coordination composition further comprises a further ligand, wherein the further ligand is a monotopic or multitopic ligand. In one embodiment, the further ligand is coordinated to one said central atom or ion. In an alternative embodiment, the further multitopic ligand is coordinated to at least two said central atoms or ions.

It is to be appreciated that the pharmaceutical compound is not particularly limited, provided it is the compound intended to be administered to a subject and is operable to form a coordinate covalent bond with the central atom or ion. In one embodiment, the pharmaceutical compound includes a functional group selected to form a coordinate covalent bond with the central atom or ion. In one embodiment the pharmaceutical compound includes a functional group which can form a coordinate bond to the central atom on ion in a monodentate or bidentate manner. In one embodiment, the functional group is carboxyl.

In one embodiment, the pharmaceutical compound comprises a nonsteroidal anti-inflammatory drug (NSAID). In one embodiment, the pharmaceutical compound is naproxen, ibuprofen, diclofenac, ketoprofen, zaltoprofen, ketorolac, chlorambucil, 5-hydroxytryptophan, L-theanine, biotin, levodopa, indomethacin or carbocysteine. In one embodiment, the pharmaceutical compound is one or more of the following (with the stippled lines indicating the coordinate covalent bond to the central atom or ion). In one embodiment, the pharmaceutical compound forms coordinate bonds to the central atom or ion in a bidentate fashion via the oxygen atom indicated by the stippled line in addition to the carbonyl oxygen atom:

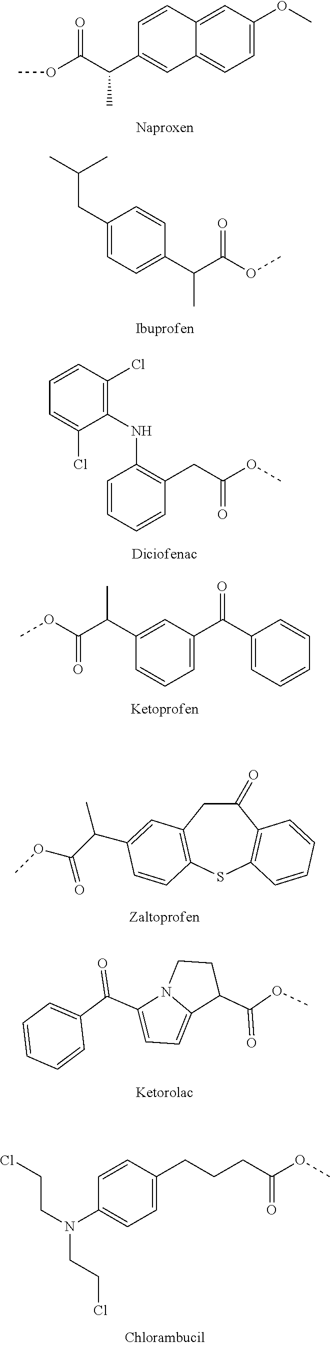

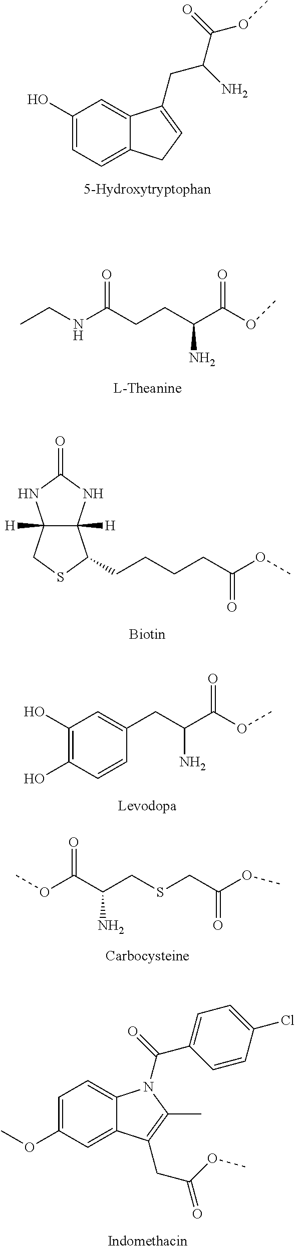

In one embodiment, the central atoms or ions comprise one or more of Zn, Cu, Mn and Mg, or one or more of Zn²⁺, Cu²⁺, Mn²⁺ and Mg²⁺; the multitopic ligand comprises one or more of 1,1′-(1,4-butanediyl)bis(imidazole), 1,1′-(1,5-pentanedidyl)bis(imidazole), 1,1′-(1,6-hexanedidyl)bis(imidazole) and 1,1′-(1,8-octanedidyl)bis(imidazole); and the pharmaceutical compound comprises one or more of diclofenac and naproxen.

In one embodiment, the central atoms or ions comprises Cu or Cu²⁺; the pharmaceutical compound comprises one or more of ibuprofen and diclofenac; and the multitopic ligand comprises one or more of the following:

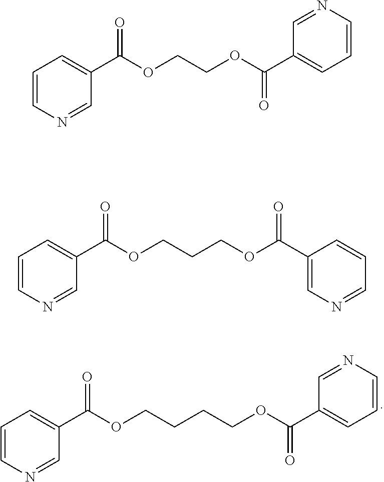 [SMILES: O=C(OCCOC(=O)c1cccnc1)c1cccnc1 ; O=C(OCCCOC(=O)c1cccnc1)c1cccnc1 ; O=C(OCCCCOC(=O)c1cccnc1)c1cccnc1]

In one embodiment, the central atoms or ions comprise Cu²⁺; the pharmaceutical compound comprises one or more of ibuprofen and diclofenac, or preferably, ibuprofen; and the multitopic ligand comprises

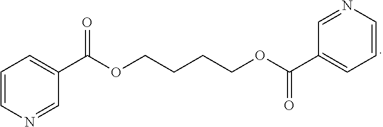 [SMILES: O=C(OCCCCOC(=O)c1cccnc1)c1cccnc1]

In one embodiment, the coordination composition is selected for forming an amorphous or semi-crystalline solid upon melting followed by cooling, wherein the amorphous or semi-crystalline solid comprises Cu²⁺, ibuprofen, niacin and 1,4-butanediol.

In one embodiment, the central atoms or ions comprise Zn or Zn²⁺; the pharmaceutical compound comprises one or more of diclofenac, ibuprofen, naproxen and indomethacin; and the multitopic ligand comprises one or more of the following:

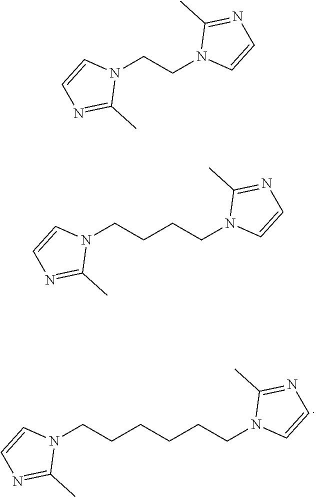 [SMILES: Cc1nccn1CCn1ccnc1C ; Cc1nccn1CCCCn1ccnc1C ; Cc1nccn1CCCCCCn1ccnc1C]

In one embodiment, the central atoms or ions comprise Zn²⁺; the pharmaceutical compound comprises one or more of diclofenac, ibuprofen, naproxen and indomethacin; and the multitopic ligand comprises

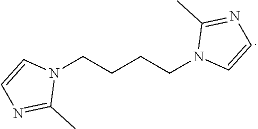 [SMILES: Cc1nccn1CCCCn1ccnc1C]

In one embodiment, the coordination composition is selected for forming an amorphous or semi-crystalline solid upon melting followed by cooling, wherein the amorphous or semi-crystalline solid is for forming a film or predetermined shape.

In one embodiment, the ligand is highly soluble in water. The applicant has appreciated that increased water solubility may permit improved controlled release of the coordinated pharmaceutical compound.

In one embodiment, the material is able to melt and upon cooling be transformed into an amorphous or semi-crystalline solid and molded into films or predetermined shapes for drug release applications. In one embodiment, the coordination composition is selected to melt upon heating and forms an amorphous or semi-crystalline solid upon cooling comprising a Cu²⁺ metal ions, ibuprofen anions, and nicotinate containing multitopic linkers.

In one embodiment, the coordination composition is for oral, intravenous, intramuscular, intrathecal, subcutaneous, parenteral, sublingual, buccal, rectal, vaginal, ocular, nasal, inhalation, optic, topical or transdermal administration. In one embodiment, the coordination composition is for oral, intravenous, ocular, parenteral, nasal, inhalation, optic, topical or transdermal administration. In one embodiment, the coordination composition is for oral administration in the form of a solution, syrup, suspension, emulsion, gel, powder, granule, capsule, or tablet. In one embodiment, the coordination composition is for rectal administration in the form of a suppository, ointment, cream, powder, or solution. In one embodiment, the coordination composition is for topical administration in the form of an ointment, cream, paste, lotion, gel, solution or topical aerosol. In one embodiment, the coordination composition is for parenteral administration in the form of an injection, implant, irrigation, or dialysis, wherein the injection preferably includes a solution, suspension or emulsion. In one embodiment, the coordination composition is for inhalation administration in the form of an aerosol, spray, or gas.

In aspect (1), there is provided a pharmaceutical coordination composition having a plurality of central atoms or ions, a multitopic ligand and one or more pharmaceutical compounds coordinated to at least one of the plurality of the central atoms or ions, wherein the multitopic ligand is coordinated to at least two of the plurality of central atoms or ions.

In aspect (2), there is provided a pharmaceutical coordination composition according to one or more of aspects (1) and (3) to (23) in any combination, wherein the coordination composition comprises a coordination polymer or macrocycle, the coordination polymer optionally being a linear coordination polymer.

In aspect (3), there is provided a pharmaceutical coordination composition according to one or more of aspects (1), (2) and (4) to (23) in any combination, wherein the coordination composition comprises a nonlinear coordination polymer, whereby at least one said central atom or ion is coupled to more than two other said central atoms or ions with the multitopic ligand interposed therebetween.

In aspect (4), there is provided a pharmaceutical coordination composition according to one or more of aspects (1) to (3) and (5) to (23) in any combination, wherein the coordination composition is a coordination polymer, each said central atom or ion having a coordination number between 3 and 8, inclusive, and at least one said central atom or ion is coordinated with one to six said pharmaceutical compounds.

In aspect (5), there is provided a pharmaceutical coordination composition according to one or more of aspects (1) to (4) and (6) to (23) in any combination, wherein each said central atom or ion in the coordination polymer has a geometry selected from the group consisting of trigonal planar, tetrahedral, square planar, trigonal bipyramidal, square pyramidal, octahedral, trigonal prismatic, pentagonal bipyramidal, capped octahedral, capped trigonal prismatic, square antiprismatic, dodecahedral, bicapped trigonal prismatic, cubic and hexagonal bipyramidal.

In aspect (6), there is provided a pharmaceutical coordination composition according to one or more of aspects (1) to (5) and (7) to (23) in any combination, wherein the central atoms or ions comprise metal or transition metal atoms or ions.

In aspect (7), there is provided a pharmaceutical coordination composition according to one or more of aspects (1) to (6) and (8) to (23) in any combination, wherein the central atoms or ions comprises one or more of Zn, Cu, Mn, Mg, Fe, Ca and Ag, or one or more of Zn²⁺, Cu²⁺, Mn²⁺, Mg²⁺, Fe²⁺, Fe³⁺, Ca²⁺ and Ag¹⁺.

In aspect (8), there is provided a pharmaceutical coordination composition according to one or more of aspects (1) to (7) and (9) to (23) in any combination, wherein the multitopic ligand comprises a plurality of coordinating portions and a linking portion interposed between the coordinating portions, wherein the plurality of the coordinating portions comprise at least first and second coordinating portions each respectively selected to form first and second coordination bonds with one and another one of the plurality of the central atoms or ions.

In aspect (9), there is provided a pharmaceutical coordination composition according to one or more of aspects (1) to (8) and (10) to (23) in any combination, wherein one or both of the first and second coordinating portions comprise an optionally substituted heterocycloalkyl or heteroaryl, wherein said heterocycloalkyl or heteroaryl is optionally selected from the group consisting of imidazolyl, methylimidazolyl, ethylimidazolyl, 1H-1,2,3-triazolyl, 4H-1,2,4-triazolyl, 1H-tetrazolyl, nicotinate and isonicotinate.

In aspect (10), there is provided a pharmaceutical coordination composition according to one or more of aspects (1) to (9) and (11) to (23) in any combination, wherein one or both of the first and second coordinating portions comprise a primary or secondary amine, or preferably, glycinamidyl.

In aspect (11), there is provided a pharmaceutical coordination composition according to one or more of aspects (1) to (10) and (12) to (23) in any combination, wherein the linking portion comprises optionally substituted branched or unbranched C₁-C₃₀ alkylene, alkenylene or alkynylene,

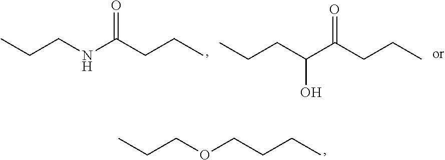

wherein one or more carbon atoms of said C₁-C₃₀ alkylene, alkenylene or alkynylene is optionally substituted with a nitrogen atom, an oxygen atom or a sulfur atom.

In aspect (12), there is provided a pharmaceutical coordination composition according to one or more of aspects (1) to (11) and (13) to (23) in any combination, The coordination composition of any one of claims 1 to 11, wherein the multitopic ligand comprises 1,1′-(1,4-butanediyl)bis(imidazole); 1,1′-(1,5-pentanedidyl)bis(imidazole); 1,1′-(1,6-hexanedidyl)bis(imidazole); 1,1′-(1,8-octanedidyl)bis(imidazole); or

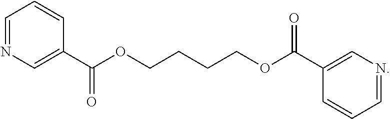 [SMILES: O=C(OCCCCOC(=O)c1cccnc1)c1cccnc1]

In aspect (13), there is provided a pharmaceutical coordination composition according to one or more of aspects (1) to (12) and (14) to (23) in any combination, wherein the multitopic ligand comprises one of the following:

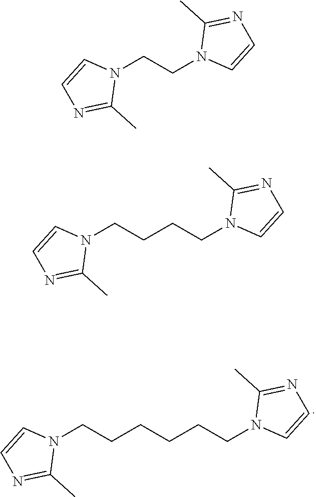 [SMILES: Cc1nccn1CCn1ccnc1C ; Cc1nccn1CCCCn1ccnc1C ; Cc1nccn1CCCCCCn1ccnc1C]

In aspect (14), there is provided a pharmaceutical coordination composition according to one or more of aspects (1) to (13) and (15) to (23) in any combination, wherein the multitopic ligand comprises one of the following:

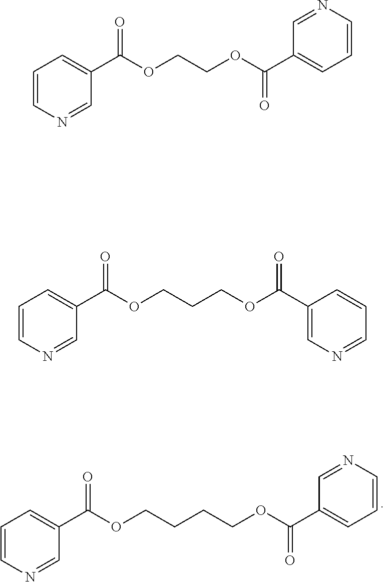 [SMILES: O=C(OCCOC(=O)c1cccnc1)c1cccnc1 ; O=C(OCCCOC(=O)c1cccnc1)c1cccnc1 ; O=C(OCCCCOC(=O)c1cccnc1)c1cccnc1]

In aspect (15), there is provided a pharmaceutical coordination composition according to one or more of aspects (1) to (14) and (16) to (23) in any combination, wherein the multitopic ligand comprises one of the following:

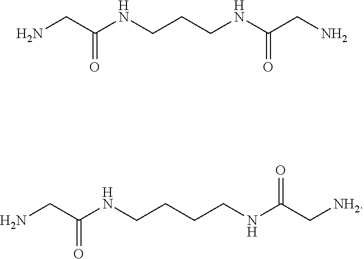 [SMILES: NCC(=O)NCCCNC(=O)CN ; NCC(=O)NCCCCNC(=O)CN]

In aspect (16), there is provided a pharmaceutical coordination composition according to one or more of aspects (1) to (15) and (17) to (23) in any combination, wherein the pharmaceutical compound comprises a carboxyl portion selected to form a coordinate covalent bond with the central atom or ion, and wherein the pharmaceutical compound is optionally selected from the group consisting of naproxen, ibuprofen, diclofenac, ketoprofen, zaltoprofen, indomethacin, ketorolac, chlorambucil, 5-hydroxytryptophan, L-theanine, biotin, levodopa and carbocysteine.

In aspect (17), there is provided a pharmaceutical coordination composition according to one or more of aspects (I) to (16) and (18) to (23) in any combination, wherein the central atoms or ions comprise one or more of Zn, Cu, Mn and Mg, or one or more of Zn²⁺, Cu²⁺, Mn²⁺ and Mg²⁺; the multitopic ligand comprises one or more of 1,1′-(1,4-butanediyl)bis(imidazole), 1,1′-(1,5-pentanedidyl)bis(imidazole), 1,1′-(1,6-hexanedidyl)bis(imidazole) and 1,1′-(1,8-octanedidyl)bis(imidazole); and the pharmaceutical compound comprises one or more of diclofenac and naproxen.

In aspect (18), there is provided a pharmaceutical coordination composition according to one or more of aspects (1) to (17) and (19) to (23) in any combination, wherein the central atoms or ions comprise Cu or Cu²⁺; the pharmaceutical compound comprises one or more of ibuprofen and diclofenac; and the multitopic ligand comprises one or more of the following:

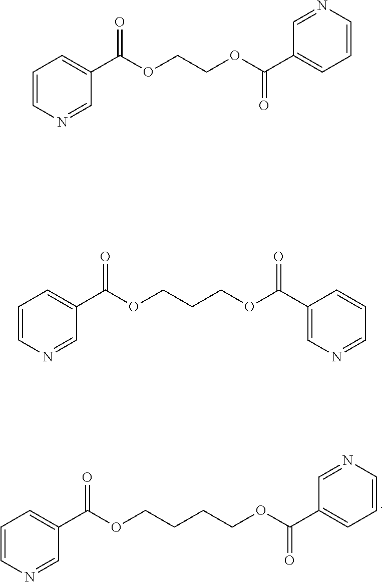 [SMILES: O=C(OCCOC(=O)c1cccnc1)c1cccnc1 ; O=C(OCCCOC(=O)c1cccnc1)c1cccnc1 ; O=C(OCCCCOC(=O)c1cccnc1)c1cccnc1]

In aspect (19), there is provided a pharmaceutical coordination composition according to one or more of aspects (1) to (18) and (20) to (23) in any combination, wherein the central atoms or ions comprise Cu²⁺; the pharmaceutical compound comprises one or more of ibuprofen and diclofenac, or preferably, ibuprofen; and the multitopic ligand comprises

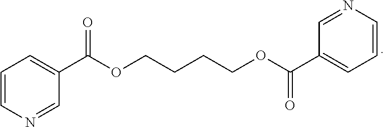 [SMILES: O=C(OCCCCOC(=O)c1cccnc1)c1cccnc1]

In aspect (20), there is provided a pharmaceutical coordination composition according to one or more of aspects (1) to (19) and (21) to (23) in any combination, wherein the coordination composition is selected for forming an amorphous or semi-crystalline solid upon melting followed by cooling, wherein the amorphous or semi-crystalline solid comprises Cu²⁺, ibuprofen, niacin and 1,4-butanediol.

In aspect (21), there is provided a pharmaceutical coordination composition according to one or more of aspects (1) to (20) and (22) to (23) in any combination, wherein the central atoms or ions comprise Zn or Zn²⁺; the pharmaceutical compound comprises one or more of diclofenac, ibuprofen, naproxen and indomethacin; and the multitopic ligand comprises one or more of the following:

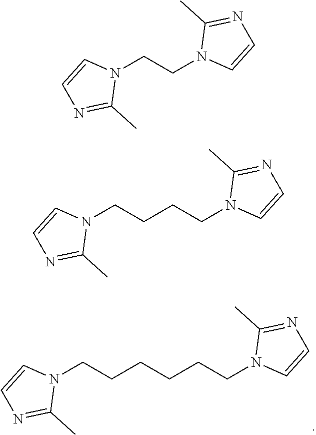 [SMILES: Cc1nccn1CCn1ccnc1C ; Cc1nccn1CCCCn1ccnc1C ; Cc1nccn1CCCCCCn1ccnc1C]

In aspect (22), there is provided a pharmaceutical coordination composition according to one or more of aspects (1) to (21) and (23) in any combination, wherein the central atoms or ions comprise Zn²⁺; the pharmaceutical compound comprises one or more of diclofenac, ibuprofen, naproxen and indomethacin; and the multitopic ligand comprises

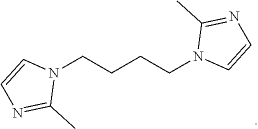 [SMILES: Cc1nccn1CCCCn1ccnc1C]

In aspect (23), there is provided a pharmaceutical coordination composition according to one or more of aspects (1) to (22) in any combination, wherein the coordination composition is selected for forming an amorphous or semi-crystalline solid upon melting followed by cooling, wherein the amorphous or semi-crystalline solid is for forming a film or predetermined shape.

In one embodiment, the term “alkyl” refers to a straight-chained or branched hydrocarbon group containing 1 to 30 carbon atoms. The alkyl may include lower alkyl, referring to a C1-C6 alkyl chain. Examples of the alkyl group include methyl, ethyl, n-propyl, isopropyl, tert-butyl, and n-pentyl. The Alkyl group may be optionally substituted with one or more substituents.

In one embodiment, the term “alkenyl” refers to an unsaturated hydrocarbon chain that may be a straight chain or branched chain, containing 2 to 30 carbon atoms and at least one carbon-carbon double bond. The Alkenyl group may be optionally substituted with one or more substituents. In one embodiment, the term “alkynyl” refers to an unsaturated hydrocarbon chain that may be a straight chain or branched chain, containing 2 to 30 carbon atoms and at least one carbon-carbon triple bond. The alkynyl groups may be optionally substituted with one or more substituents. The sp² or sp carbons of the alkenyl or alkynyl group may optionally be the point of attachment of the group.

In one embodiment, the term “heterocycloalkyl” refers to a nonaromatic 5-8 membered monocyclic, 8-12 membered bicyclic or 11-14 membered tricyclic ring system comprising 1-3 heteroatoms if monocyclic, 1-6 heteroatoms if bicyclic or 1-9 heteroatoms if tricyclic, said heteroatoms being O, N, S, B, P or Si. The heterocycloalkyl is optionally substituted with one or more substituents. In one embodiment, the heterocycloalkyl may include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl or thiirene.

In one embodiment, the term “heteroaryl” refers to an aromatic 5-8 membered monocyclic, 8-12 membered bicyclic or 11-14 membered tricyclic ring system having 1-4 ring heteroatoms if monocyclic, 1-6 heteroatoms if bicyclic or 1-9 heteroatoms if tricyclic, where the heteroatoms are independently O, N or S, and the remainder ring atoms are carbon. The heteroaryl is optionally substituted with one or more substituents. In one embodiment, the heteroaryl is pyridyl, 1-oxo-pyridyl, furanyl, benzo[1,3]dioxolyl, benzo[1,4]clioxinyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo[b]thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl or pyrrolo[2,1 b]oxazolyl.

In one embodiment, the term “substituent” or “substituted” means that a hydrogen atom is replaced with a group that does not substantially adversely affect the stability or activity of the compound. The term “substituted” refers to one or more substituents, which may be the same or different, each replacing a hydrogen atom. In one embodiment, the substituent is halogen, hydroxyl, amino, alkylamino, arylamino, dialkylamino, diarylamino, cyano, nitro, mercapto, oxo, carbonyl, thio, imino, formyl, carbamido, carbamyl, carboxyl, thioureido, thiocyanato, sulfoamido, sulfonylalkyl, sulfonylaryl, alkyl, alkenyl, alkoxy, mercaptoalkoxy, aryl, heteroaryl, cyclyl, heterocyclyl, wherein alkyl, alkenyl, alkyloxy, aryl, heteroaryl, cyclyl and heterocyclyl are optionally substituted with alkyl, aryl, heteroaryl, halogen, hydroxyl, amino, mercapto, cyano, nitro, oxo, thioxo or imino.

Additional and alternative features of the present invention will be apparent to a person skilled in the art from the following detailed description of the preferred embodiments thereof.

### BRIEF DESCRIPTION OF THE DRAWINGS

Reference may now be had to the following detailed description taken together with the accompanying drawings in which:

FIG. 1 shows structural formulas of naproxen, ibuprofen and diclofenac, as well as sodium or potassium salts of the foregoing;

FIG. 2 shows possible one-dimensional and two-dimensional arrangements of coordination polymers in accordance with preferred embodiments of the present invention;

FIG. 3 shows structural formulas of bis-imidazole ligands in accordance with preferred embodiments of the present invention;

FIG. 4 shows the molecular structure of a magnesium coordination polymer in accordance with a preferred embodiment of the present invention, and as determined by single crystal X-ray diffraction, where the polymer included a Biim-6 linker (grey carbon bonds) and diclofenac as the pharmaceutical anion (gold carbon bonds);

FIG. 5 shows a dot graph illustrating dissolution, in 0.05 M phosphate buffer at pH 6.8, of Zn-diclofenac polymers/macrocycles prepared with bis-imidazole ligands of different lengths or sizes in accordance with preferred embodiments of the present invention, and which includes as the X-axis time in hours and the Y-axis percent diclofenac released;

FIG. 6 shows a dot graph illustrating dissolution, in 0.05 M phosphate buffer at pH 6.8, of Zn—, Mg— and Mn-diclofenac polymers/macrocycles prepared with bis-imidazole ligands of different lengths or sizes in accordance with preferred embodiments of the present invention, and which includes as the X-axis time in hours and the Y-axis percent diclofenac released;

FIG. 7 shows various components of coordination polymers in accordance with a preferred embodiment of the present invention;

FIG. 8 shows a dissolution curve of Cu(diclo)₂(biim4) and Cu(diclo)₂(nic4) in 0.01M citrate buffer+0.5% SDS;

FIG. 9 shows a structure of the TCP Cu(ibuprofen)₂(nic4)(H₂O)₂ obtained from single-crystal X-ray diffraction;

FIG. 10 shows optical microscopy images of Cu(ibuprofen)₂(nic4)(H₂O)₂ between temperatures of 30.0° C.-90.0° C.;

FIG. 11 shows a Differential Scanning Calorimetry profile of Cu(ibuprofen)₂(nic4)(H₂O)₂ between temperatures of −50° C.-95° C. at a ramp rate of 2°/min.;

FIG. 12 shows powder X-ray diffraction patterns of conversion of Cu(ibuprofen)₂(nic4)(H₂O)₂ to a new crystalline phase Cu(ibuprofen)₄(nic4) at temperatures between 30° C.-100° C.;

FIG. 13 shows Cu(ibuprofen)₂(nic4)(H₂O)₂ powder at 30° C., 70° C., and 100° C.;

FIG. 14 shows structure of the TCP Cu₂(ibuprofen)₄(nic4) obtained from single crystal X-ray crystallography;

FIG. 15 shows a Differential Scanning Calorimetry profile of Cu₂(ibuprofen)₄(nic4) between temperatures of −50° C.-160° C. at a ramp rate of 2°/min.;

FIG. 16 shows X-Ray powder diffraction patterns of Cu₂(ibuprofen)₄(nic4) and Cu(ibuprofen)₂(nic4)(H₂O)₂ at 70° C.;

FIG. 17 shows, on the left, crystal structure of Zn(diclofenac)₂(biim-4m) and, on the right, its one-periodic structure;

FIG. 18 shows, on the left, crystal structure of Zn(ibuprofen)₂(biim-4m) and, on the right, its one-periodic structure;

FIG. 19 shows, on the left, crystal structure of Zn(indomethacin)₂(biim-4m) and, on the right, its one-periodic structure;

FIG. 20 shows, on the left, crystal structure of Zn(naproxen)₂(biim-4m) and, on the right, its one-periodic structure;

FIG. 21 shows dissolution rate of sodium diclofenac (left) and Zn(diclofenac)₂(biim-4m) (right) in 0.05M phosphate buffer pH 6.8, where sodium diclofenac was 100% released after 1 h, and Zn(diclofenac)₂(biim-4m) released ˜30% of the API (diclofenac) after 72h;

FIG. 22 shows intrinsic dissolution rate (IDR) of sodium diclofenac (triangle) and Zn(diclofenac)₂(biim-4m) (square) in 0.05M phosphate buffer pH 6.8, where the IDR of sodium diclofenac was 1161±0.216 μg cm⁻² min⁻¹, and the IDR of Zn(diclofenac)₂(biim-4m) was 4.8±0.089 μg cm⁻² min⁻¹;

FIG. 23 shows dissolution rate of sodium ibuprofen (triangle) and Zn(ibuprofen)₂(biim-4m) (square) in 0.05M phosphate buffer pH 6.8, where sodium ibuprofen was 100% released after 5 min, and Zn(ibuprofen)₂(biim-4m) released 100% of the API (ibuprofen) after about 20h;

FIG. 24 shows intrinsic dissolution rate (IDR) of Zn(ibuprofen)₂(biim-4m) in 0.05M phosphate buffer pH 6.8, which was 46.5±0.006 μg cm⁻² min⁻¹;

FIG. 25 shows dissolution rate of Zn(indomethacin)₂(biim-4m) in 0.05M phosphate buffer pH 6.8, where Zn(indomethacin)₂(biim-4m) released ˜40% of the API (indomethacin) after about 72h;

FIG. 26 shows TGA profile of Zn(diclofenac)₂(biim-4m), where the temperature onset for degradation of the TCP was 255° C.;

FIG. 27 shows TGA profile of Zn(ibuprofen)₂(biim-4m), where the temperature onset for degradation of the TCP was 343° C.;

FIG. 28 shows TGA profile of Zn(indomethacin)₂(biim-4m), where the temperature onset for degradation of the TCP was 328° C.; and

FIG. 29 shows TGA profile of Zn(naproxen)₂(biim-4m), where the temperature onset for degradation of the TCP was 316° C.

### DETAILED DESCRIPTION OF THE PREFERRED EMBODIMENTS

It has been envisioned that in a preferred embodiment, the pharmaceutical coordination composition may have the following components:

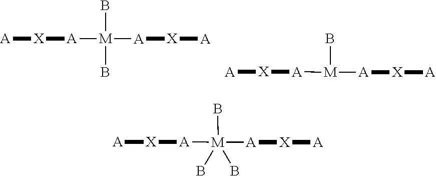

“M” represents a central metal ion, “B” represents a pharmaceutical compound and “A-X-A” represents a ligand, where “A” is a coordinating portion and “X” is a linking portion. The central metal ion is shown above with two coordinate bonds to two coordinating portions of two separate ligands and one to three pharmaceutical compounds coordinated thereto. While not shown, it has been envisioned that the remaining “A” not shown coordinated to “M” may form a coordinate bond with another “M”, so as to form a polymer or macrocycle structure. “M” may also be coordinated to solvent molecules, such as water or ethanol.

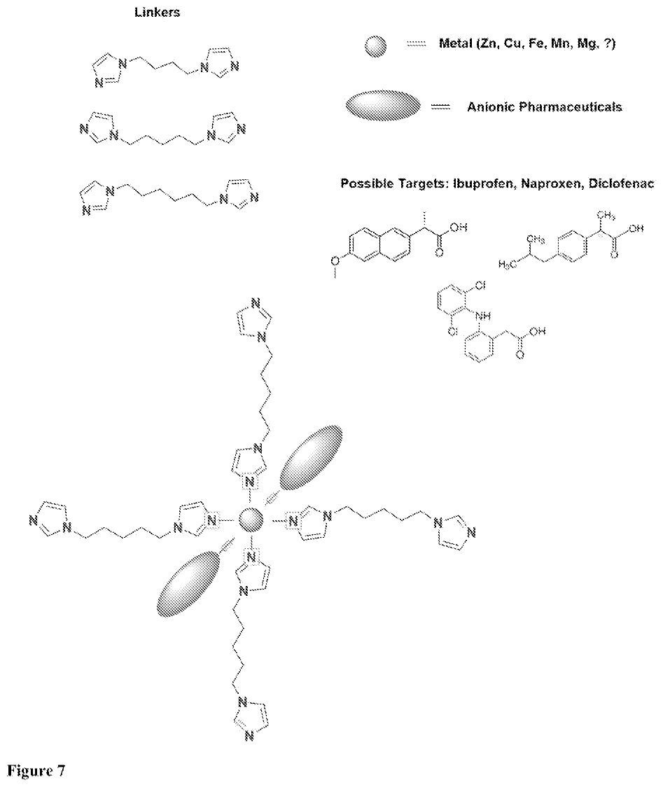

The coordination composition may be prepared by mixing a central atom or ion, a multitopic ligand and a pharmaceutical compound in a solution, which may preferably include an alcohol, such as ethanol. In one example, the pharmaceutical compound, metal salt and ligand were dissolved in a solvent, combined and precipitated from a solution. This occurred with or without heating or cooling the solution, and may be conducted at room temperature under ambient conditions. The pharmaceutical compound was either in free acid or free base form (such as a sodium or potassium salt). The metal salt could be a metal cation such as Zn(II), Cu(II), Mn(II), Mg(II), Fe(II), Fe(III), Ca(II) and/or Ag(I), with various anions, such as Cl, Br, NO₃, CO₃, SO₄, BF₄, CF₃SO₃ and others. The solvents may be but not limited to: methanol, ethanol, water, acetonitrile, dimethyl sulfoxide, nitromethane, isopropyl alcohol, dimethylformamide or a mixture thereof. The coordination polymer then grew over a period of time that ranged from seconds up to one month. The rate at which the coordination polymers grow is dependent on the concentration of the solutions used in the synthesis. FIG. 7 shows various components of a preferred coordination composition or polymer, which may be formed.

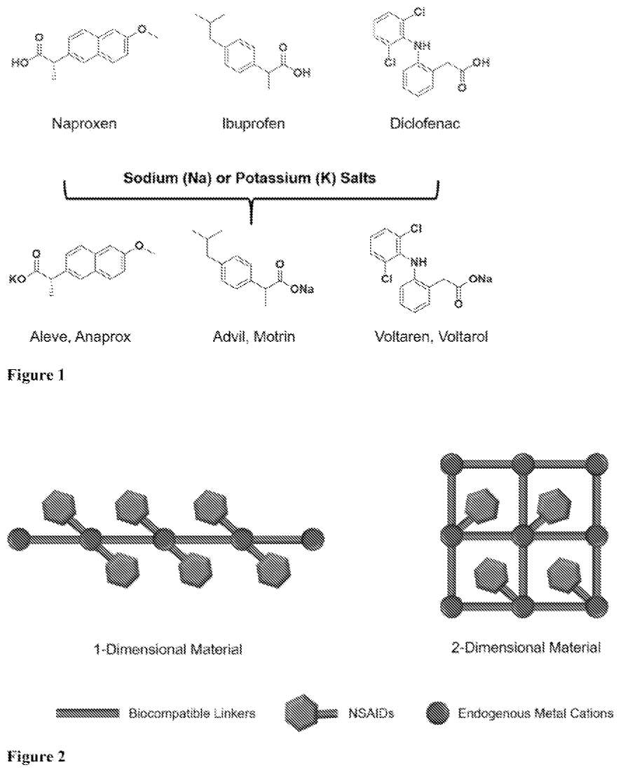

Experimental studies were conducted with three pharmaceuticals, or namely, Naproxen, Ibuprofen, and Diclofenac, with the structural formulas thereof illustrated in FIG. 1. It has been appreciated that in the free acid form, these drugs have poor uptake in the body because of poor solubility, and to make these drugs more available for uptake by our bodies, they have been sold as sodium (Na) or potassium (K) salts that are more soluble than the free acid form. In some cases, the increase in solubility may lead to the ‘burst effect’ where a large quantity of the medication is released very quickly, possibly leading to poor uptake of the drug, because most of it may be metabolized in the stomach and can cause side effects such as gastrointestinal issues.

It has been appreciated that the coordination composition of the present invention may permit prolonged or controlled release of pharmaceuticals and thus improved patient compliance and reduced side effects.

Experimental studies were conducted with compositions prepared in accordance with preferred embodiments of the present invention, and which include pharmaceuticals combined with endogenous metals (Zn, Mg, etc.) with biocompatible organic linkers to create therapeutic coordination polymers (TCPs). These materials can be 1-dimensional or 2-dimensional, as seen in FIG. 2. The pharmaceutical was coordinated to a metal cation which is propagated by the organic linker. Whether or not the material is 1-dimensional or 2-dimensional is dependent on the metal used and the coordination environment. In the studies conducted, most of the TCPs were indicated to be 1-dimensional.

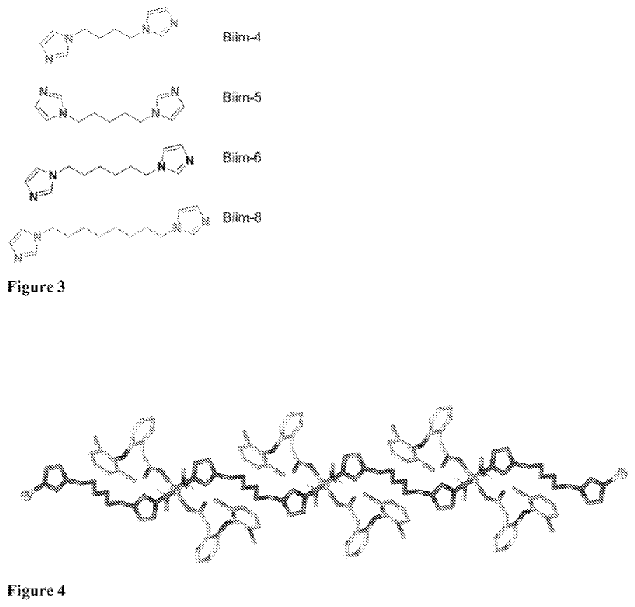

The biocompatible linkers used in the studies conducted are: 1,1′-(1,4-butanediyl)bis(imidazole) (biim-4); 1,1′-(1,5-pentanedidyl)bis(imidazole) (biim-5); 1,1′-(1,6-hexanedidyl)bis(imidazole) (biim-6); and 1,1′-(1,8-octanedidyl)bis(imidazole) (biim-8) as shown in FIG. 3. Synthesis of TCPs were conducted with a solution of bis-imidazole ligand (1 mmol) added to a solution of pharmaceutical (0.5 mmol) and stirred. A solution of metal salt (0.2-0.25 mmol) was added dropwise with stirring to the ligand-drug solution.

To prepare TCPs where the drug is a free acid there is a drug:organic ligand:metal molar ratio of 4:2:1.25 or 2:2:1.25. Typically, a solution of ligand is added to a solution of drug and stirred. Then a solution of metal salt is added dropwise with stirring to the ligand-drug solution. In the cases where the sodium or potassium salt of the drug in the synthesis, including where we deprotonate the free acid drug ourselves with sodium or potassium hydroxide, the molar ratio of drug:organic linker:metal is 2:2:1.25 or 1:2:1.25. The procedure is the same as the one with the free acid where the drug and linker are first mixed in solution and the metal salt is added to the resulting mixture. The coordination polymers then grow over a period that ranges from seconds up to one week. The rate at which the coordination polymer grows is dependent on the concentration of the solutions (drug, linker, and metal salt) that were used in the synthesis. The solvents that can be used to make the coordination polymers are methanol, ethanol, water, acetonitrile, dimethyl sulfoxide, nitromethane, isopropyl alcohol, and dimethylformamide. Coordination polymers can be synthesized using only one solvent or a mixture of solvents. For example, the drug, linker and metal salt are all dissolved in ethanol, or the drug is dissolved in water, the linker dissolved in methanol, and the metal salt dissolved in acetonitrile. All TCPs reported were made with methanol or ethanol. After 24 hours, crystals were washed with cold methanol or ethanol. An example of a typical TCP is shown in FIG. 4.

It has been appreciated that the pharmaceutical loading in the TCPs may be less than that of a commercially available pharmaceutical sodium salt due to the mass of the organic linkers. Provided below is Table I showing a summary for the relative percentages in some of specific diclofenac TCPs. For example, the percent of diclofenac in sodium diclofenac is 93%, whereas the TCPs had loading ranging from 42-70%. The percentage of metal within the TCPs by mass ranged from 2.6-7.9% and the percent of linker by mass ranged from 22-41%. The TCP with EtOH in the name were synthesized in EtOH, all others were synthesized in MeOH. However, it is not strictly required that TCPs be prepared in MeOH and TCPs may be prepared in EtOH instead, or other solvents such as but not limited to: water, acetonitrile, dimethyl sulfoxide, nitromethane, isopropyl alcohol, dimethylformamide or a mixture thereof.

TABLE 1
SUMMARY OF RELATIVE PERCENTAGE OF DICLOFENAC,
LINKER, AND METAL IN DICLOFENAC TCPs.

| | 1D or 2D or | Percent | Percent | Percent |
|---|---|---|---|---|
| TCP | Macrocycle | Diclofenac | Linker | Metal |
| Dic Zn 4 | Macrocycle | 70 | 22 | 7.7 |
| Dic Zn 5 | 1D | 69 | 24 | 7.6 |
| Dic Zn 6 | 1D | 65 | 24 | 7.2 |
| Dic Zn 6 EtOH | 1D | 64 | 24 | 7.1 |
| Dic Zn 8 | 1D | 65 | 27 | 7.2 |
| Dic Cu 4 | 1D | 70 | 23 | 7.5 |
| Dic Cu 5 | 1D | 69 | 24 | 7.4 |
| Dic Cu 6 | 1D | 63 | 23 | 6.8 |
| Dic Mn 4 | 2D | 42 | 41 | 7.9 |
| Dic Mn 6 | 1D | 64 | 24 | 5.9 |
| Dic Mn 6 EtOH | 1D | 62 | 23 | 5.7 |
| Dic Mg 6 | 1D | 66 | 24 | 2.7 |
| Dic Mg 6 EtOH | 1D | 64 | 24 | 2.6 |

Previously, pharmaceutical salts released completely in 0.05 M phosphate buffer (pH 6.8) after one hour. The TCPs synthesized in the studies were shown to have varying rates of release that depends on (1) the length of the organic linker and (2) the metal cation present.

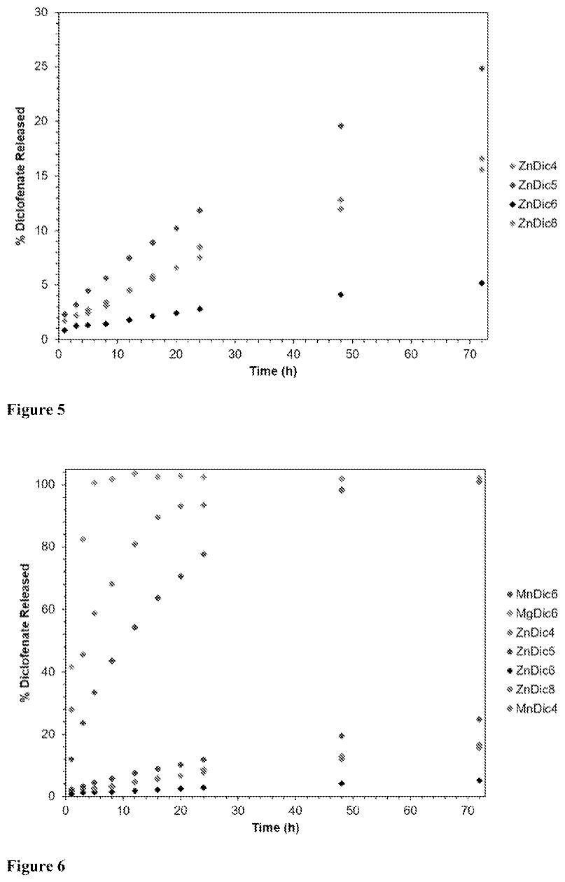

As seen in FIG. 3, between the Biim linkers is the number of carbon atoms between the two imidazole moieties. To show the effect of linker length on the release of diclofenac, four TCPs with Zn and diclofenac present were tested. The only difference was the length of the linker and it was shown that the change in length may have an effect in the release rate of diclofenac. For instance, FIG. 5 shows the release of the four TCPs in phosphate buffer. The TCPs are named based on the metal (Zn), drug (Dic=Diclofenac), and linker (4=Biim-4). The graph seen in FIG. 5 shows that out of the four TCPs, ZnDic6 had the slowest release with only 5% released after three days, whereas ZnDic5 released about 5 times that of ZnDic6 after the same amount of time. ZnDic8 and ZnDic4 had similar release. It has been experimentally proven that ZnDic4 can form a macrocycle and not a coordination polymer, permitting for the difference in diclofenac release. Experimental studies were performed with ZnNap4 (Zn, naproxen, Biim-4), a TCP, under the same conditions and the naproxen release reached 40% after three days. It may be possible that the foregoing may result from naproxen being more soluble than diclofenac and/or the pharmaceutical releasing more efficiently in a polymer form.

The applicant has appreciated that the metal cation used to make the TCP may have a significant effect on the release of diclofenac. Experimental studies have been conducted with Mn, Mg, Zn, and Cu, however, the metal atom or ion is not restricted to the foregoing and may include other metal atoms and ions, such as Fe, Co, Cr, and Gd. As seen in FIG. 6, compared with Zn TCPs, Mg and Mn TCPs shower more rapid release. MgDic6 released the quickest with complete release in 5 hours. The Mn TCPs released completely after three days and release more than all the Zn TCPs after 72 hours, in 1-3 hours.

In another study, TCPs with ester-based nicotinate coordinating groups with various length alkyl chain spacers were prepared, including those identified below. The applicant has appreciated the ability for ester-based compounds (linkers) to degrade through known mechanisms in the body, and in this case, result in the release of compounds with low toxicity (i.e. niacin (vitamin B₃) and diols such as 1,3-propanediol) which may be advantageous for

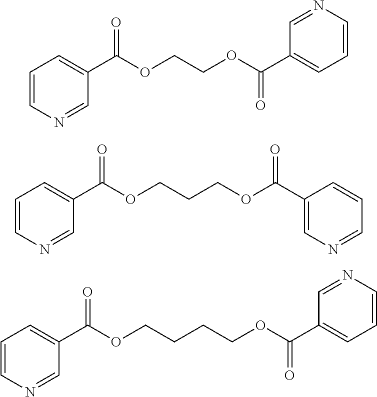

controlled drug release materials.
Nicotinate-based linkers, such as (nic4), were synthesized according to known literature procedures (J. Am. Chem. Soc. 2004, 126, 34, 10645-10656, which is incorporated herein by reference). TCPs with nicotinate-based linkers were made by dissolving the nicotinate-based linker in a mixture of ethanol, methanol, acetonitrile, and/or water, such as methanol/ethanol/water (5:5:1). To this solution, a desired pharmaceutical (such as diclofenac, sodium ibuprofen, etc.) was then added, in addition to the desired metal salt (such as Cu(NO₃)₂). This solution was left to sit at room temperature until crystalline material precipitated from solution, often in less than 24h.

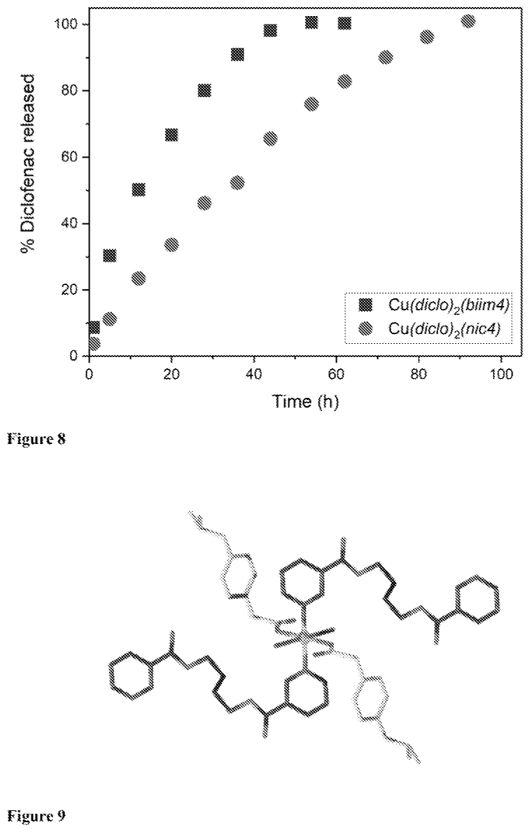

The nicotinate-based TCPs showed different release profiles of pharmaceutical compounds compared to imidazole-based TCPs of a similar type. Such an example is shown with FIG. 8 which demonstrates different release profiles of Cu(II) TCPs containing the same pharmaceutical (diclofenac) with nicotinate or imidazole-based linkers both with four carbon alkyl chains between the terminal coordinating groups.

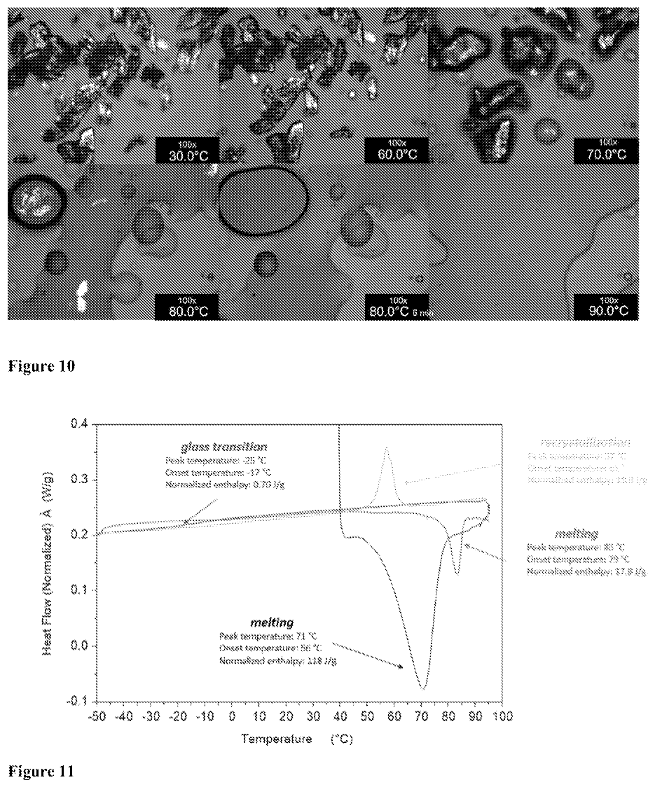

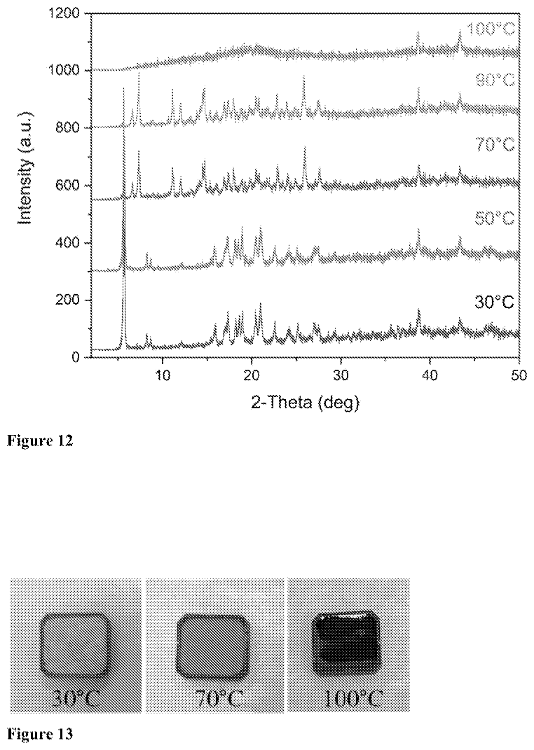

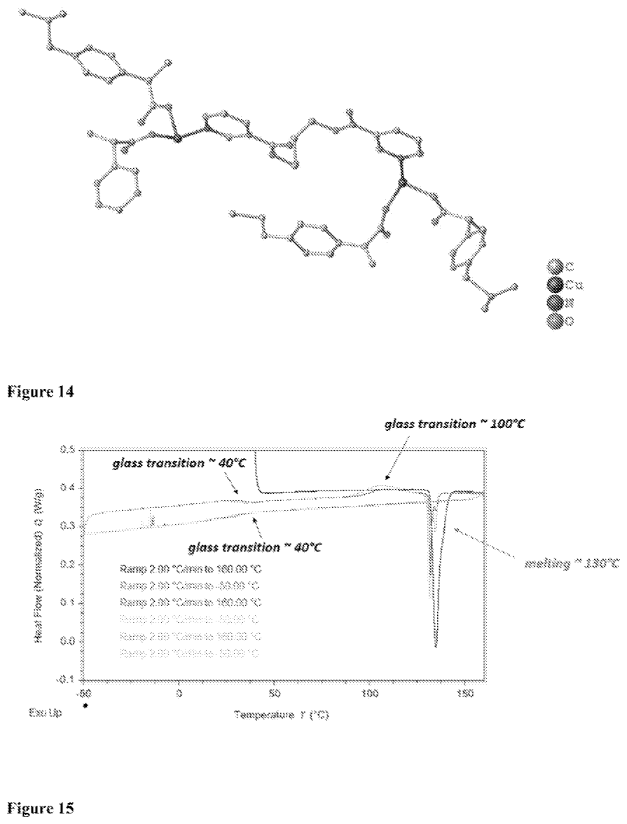

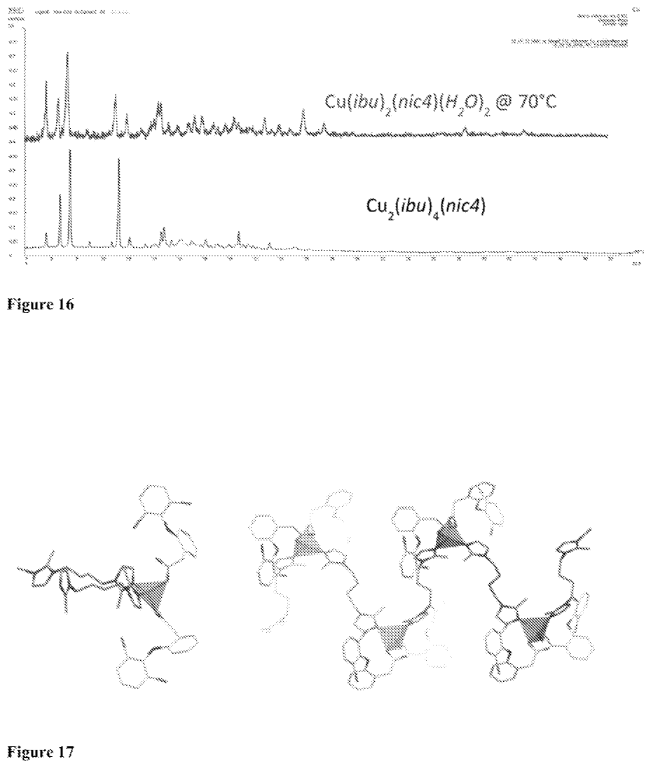

Cu-based TCPs with nicotinate linkers and ibuprofen pharmaceutical compounds were prepared, which showed the ability to melt and form amorphous glass-type solids or new crystalline solids based on defined heating and cooling rates. In one example, the melting point of a Cu(II) nicotinate-based TCP with ibuprofen pharmaceutical compounds had a melting point between 50° C. and 80° C. and could be subsequently transformed into a different crystalline solid form. In this particular example, Cu(ibuprofen)₂(nic4)(H₂O)₂ melted at low temperatures (between 50° C.-80° C.), as seen in FIG. 10, and converts to diatomic Cu₂(ibuprofen)₄(nic4) which has a diatomic Cu(II) ‘paddlewheel’ topology. This conversion was confirmed by single crystal and powder X-ray crystallography experiments, as seen in FIGS. 9, 12, 14 and 16, as well as with images seen in FIG. 13. Differential scanning calorimetry profiles of Cu(ibuprofen)₂(nic4)(H₂O)₂ and Cu₂(ibuprofen)₄(nic4) are respectively seen in FIGS. 11 and 15. Cu₂(ibuprofen)₄(nic4) also exhibited melting behaviour that occurs at temperatures between 120° C.-140° C. The applicant has appreciated that the ability to melt TCPs and process them into defined shapes or films as desired may permit suitable medical applications.

In another study, TCPs with methylimidazole-based linkers were prepared, which are identified below:

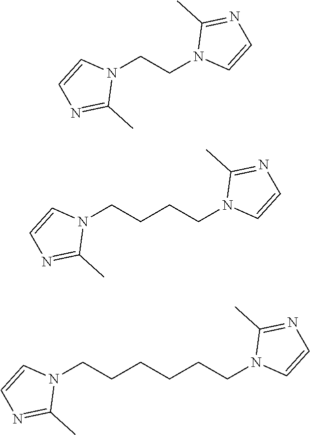 [SMILES: Cc1nccn1CCn1ccnc1C ; Cc1nccn1CCCCn1ccnc1C ; Cc1nccn1CCCCCCn1ccnc1C]

Methylimidazole-based linkers were prepared by adapting a literature procedure (DOI: 10.1039/c9sc03760h or Chem. Sci., 2019, 10, 9998-10002, which are incorporated herein by reference). Methylimidazole-based TCPs were made by adding a solution of the bis-methylimidazole ligand (1 mmol) in ethanol, methanol, or water, to a solution of the drug (diclofenac, sodium ibuprofen, indomethacin, naproxen) in ethanol, methanol, or water (1-0.5 mmol) and stirred. A solution of the metal salt (zinc nitrate) in acetonitrile, ethanol, methanol, or water (0.5-0.25 mmol) was added dropwise with stirring to ligand-drug solution. After 24-48 h, clear colourless crystals were dried at room temperature.

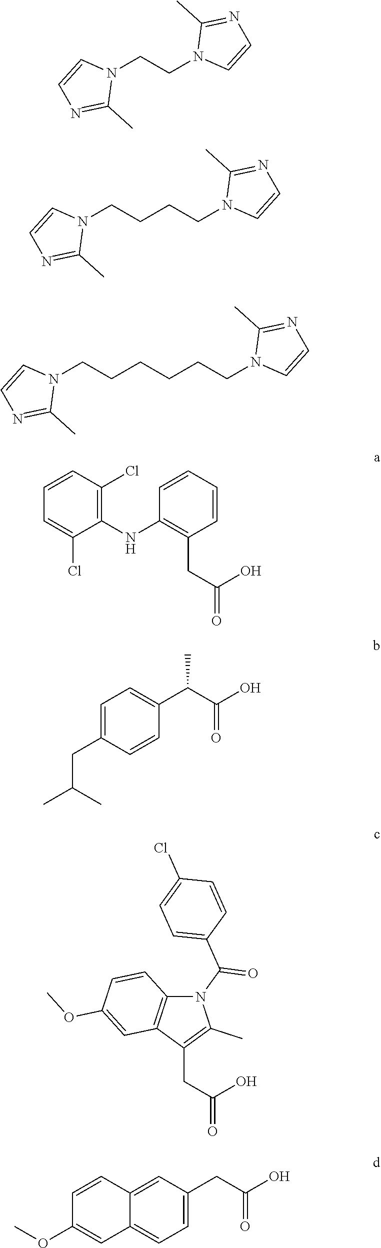

Shown above are examples of bis-methylimidazole organic linkers and anionic pharmaceuticals used for the construction of TCPs: a) diclofenac, b) ibuprofen, c) indomethacin, and d) naproxen.

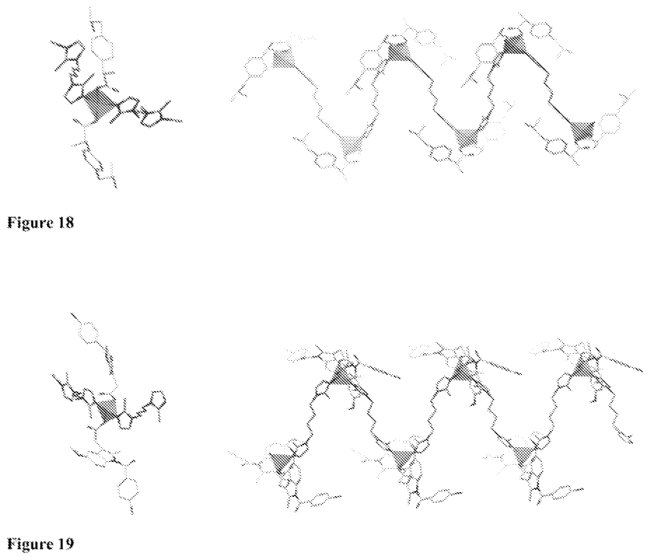

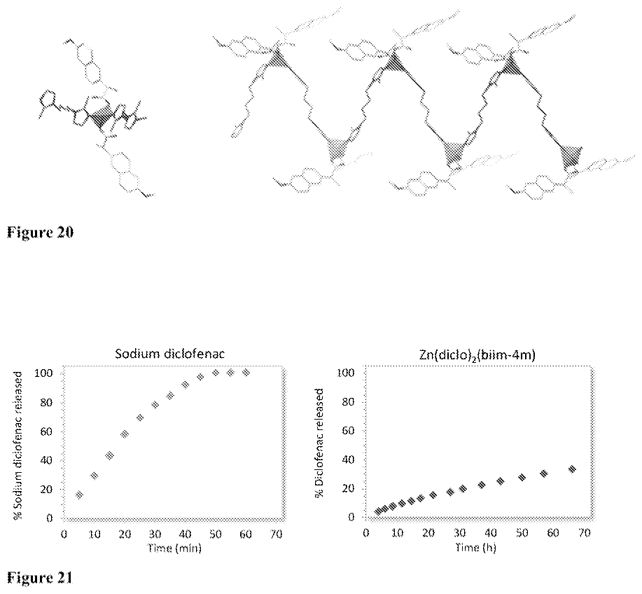

FIGS. 17 to 20 show crystal and one-periodic structures respectively of Zn(diclofenac)₂(biim-4m), Zn(ibuprofen)₂(biim-4m), Zn(indomethacin)₂(biim-4m) and Zn(naproxen)₂(biim-4m).

The methylimidazole linker was shown to form TCPs more readily with various NSAIDs, including indomethacin for the first time. Although not intended to be bound by theory, it is possible that compared to an imidazole linker, increased steric bulk may facilitate crystallization and increase release rates of pharmaceuticals when incorporated into TCPs. It is also possible that the methylimidazole multitopic ligand with a butane linking portion (Biim-4m) may affect the chain propagation length, with Biim-4 leading to dimeric macrocycle formation in the presence of diclofenac anions, whereas Biim-4m led to periodic coordination polymers. It has been appreciated that the ability to control the chain propagation length by altering the organic linker may have significant impact on the dissolution rate of drugs from TCPs.

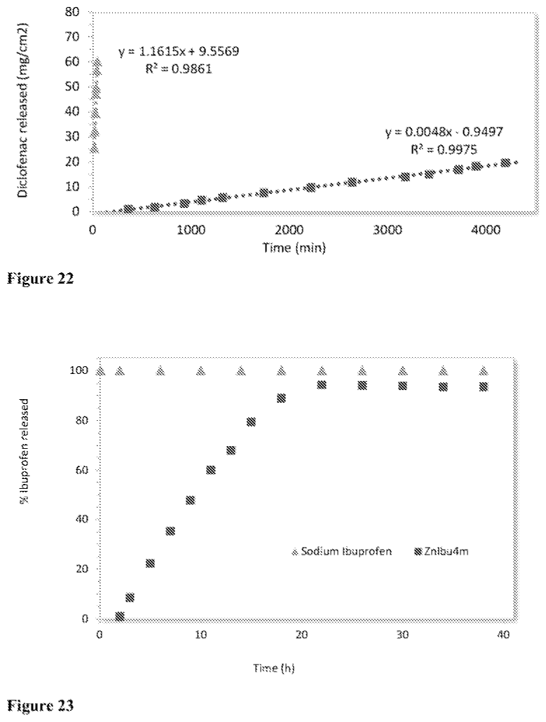

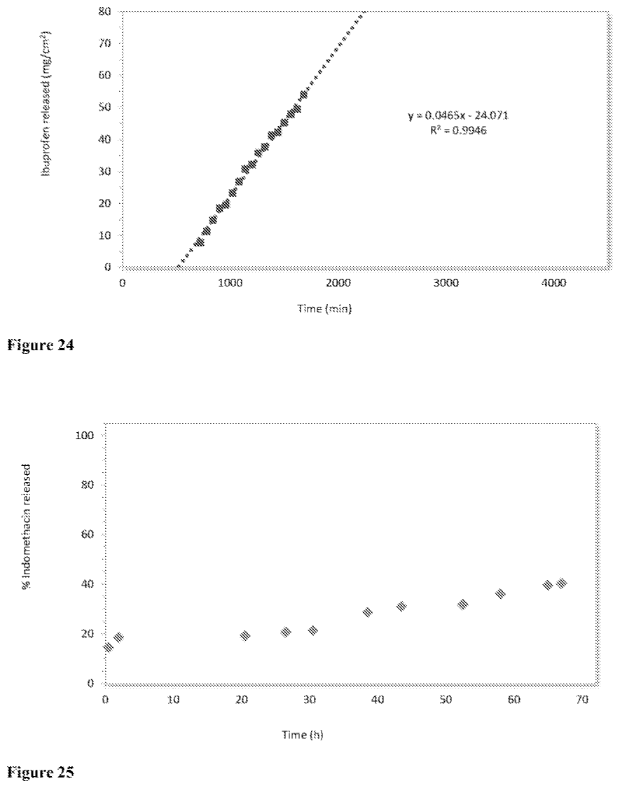

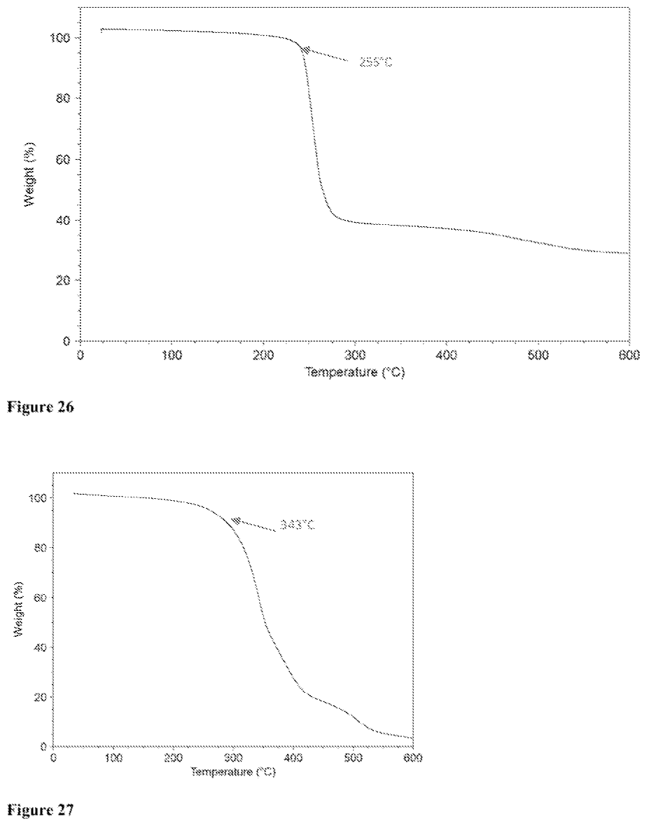

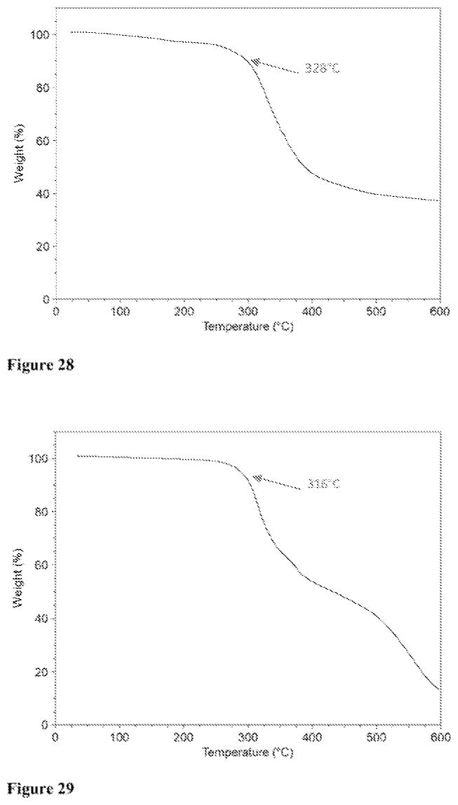

FIGS. 21 to 25 show results of dissolution experiments performed with sodium diclofenac, Zn(diclofenac)₂(biim-4m), sodium ibuprofen, Zn(ibuprofen)₂(biim-4m) and Zn(indomethacin)₂(biim-4m). FIGS. 26 to 29 show TGA profiles of Zn(diclofenac)₂(biim-4m), Zn(ibuprofen)₂(biim-4m), Zn(indomethacin)₂(biim-4m) and Zn(naproxen)₂(biim-4m).

While the invention has been described with reference to preferred embodiments, the invention is not or intended by the applicant to be so limited. A person skilled in the art would readily recognize and incorporate various modifications, additional elements and/or different combinations of the described components consistent with the scope of the invention as described herein.

1. A pharmaceutical coordination composition having a plurality of central atoms or ions, a multitopic ligand and one or more pharmaceutical compounds coordinated to at least one of the plurality of the central atoms or ions, wherein the multitopic ligand is coordinated to at least two of the plurality of central atoms or ions.

2. The coordination composition of claim 1, wherein the coordination composition comprises a coordination polymer or macrocycle, the coordination polymer optionally being a linear coordination polymer.

3. The coordination composition of claim 1, wherein the coordination composition comprises a nonlinear coordination polymer, whereby at least one said central atom or ion is coupled to more than two other said central atoms or ions with the multitopic ligand interposed therebetween.

4. The coordination composition of claim 1, wherein the coordination composition is a coordination polymer, each said central atom or ion having a coordination number between 3 and 8, inclusive, and at least one said central atom or ion is coordinated with one to six said pharmaceutical compounds.

5. The coordination composition of claim 1, wherein each said central atom or ion in the coordination polymer has a geometry selected from the group consisting of trigonal planar, tetrahedral, square planar, trigonal bipyramidal, square pyramidal, octahedral, trigonal prismatic, pentagonal bipyramidal, capped octahedral, capped trigonal prismatic, square antiprismatic, dodecahedral, bicapped trigonal prismatic, cubic and hexagonal bipyramidal.

6. The coordination composition of claim 1, wherein the central atoms or ions comprise metal or transition metal atoms or ions.

7. The coordination composition of claim 1, wherein the central atoms or ions comprises one or more of Zn, Cu, Mn, Mg, Fe, Ca and Ag, or one or more of Zn²⁺, Cu²⁺, Mn²⁺, Mg²⁺, Fe²⁺, Fe³⁺, Ca²⁺ and Ag¹⁺.

8. The coordination composition of claim 1, wherein the multitopic ligand comprises a plurality of coordinating portions and a linking portion interposed between the coordinating portions, wherein the plurality of the coordinating portions comprise at least first and second coordinating portions each respectively selected to form first and second coordination bonds with one and another one of the plurality of the central atoms or ions.

9. The coordination composition of claim 1, wherein one or both of the first and second coordinating portions comprise an optionally substituted heterocycloalkyl or heteroaryl, wherein said heterocycloalkyl or heteroaryl is optionally selected from the group consisting of imidazolyl, methylimidazolyl, ethylimidazolyl, 1H-1,2,3-triazolyl, 4H-1,2,4-triazolyl, 1H-tetrazolyl, nicotinate and isonicotinate.

10. The coordination composition of claim 1, wherein one or both of the first and second coordinating portions comprise a primary or secondary amine, or preferably, glycinamidyl.

11. The coordination composition of claim 1, wherein the linking portion comprises optionally substituted branched or unbranched C₁-C₃₀ alkylene, alkenylene or alkynylene, 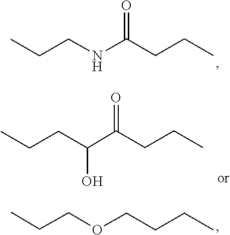
wherein one or more carbon atoms of said C₁-C₃₀ alkylene, alkenylene or alkynylene is optionally substituted with a nitrogen atom, an oxygen atom or a sulfur atom.

12. The coordination composition of claim 1, wherein the multitopic ligand comprises 1,1′-(1,4-butanediyl)bis(imidazole); 1,1′-(1,5-pentanedidyl)bis(imidazole); 1,1′-(1,6-hexanedidyl)bis(imidazole); 1,1′-(1,8-octanedidyl)bis(imidazole); or

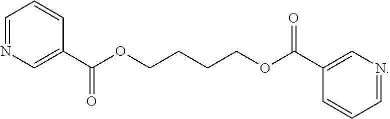 [SMILES: O=C(OCCCCOC(=O)c1cccnc1)c1cccnc1]

13. The coordination composition of claim 1, wherein the multitopic ligand comprises one of the following:

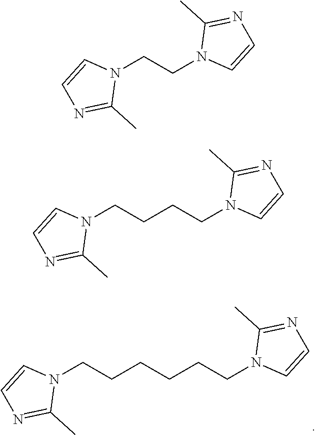 [SMILES: Cc1nccn1CCn1ccnc1C ; Cc1nccn1CCCCn1ccnc1C ; Cc1nccn1CCCCCCn1ccnc1C]

14. The coordination composition of claim 1, wherein the multitopic ligand comprises one of the following:

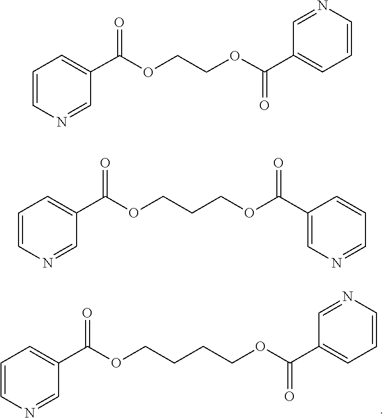

15. The coordination composition of claim 1, wherein the multitopic ligand comprises one of the following:

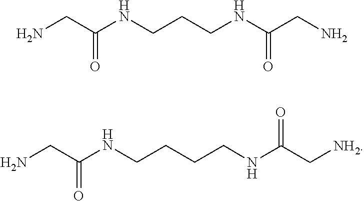 [SMILES: NCC(=O)NCCCNC(=O)CN ; NCC(=O)NCCCCNC(=O)CN]

16. The coordination composition of claim 1, wherein the pharmaceutical compound comprises a carboxyl portion selected to form a coordinate covalent bond with the central atom or ion, and wherein the pharmaceutical compound is optionally selected from the group consisting of naproxen, ibuprofen, diclofenac, ketoprofen, zaltoprofen, indomethacin, ketorolac, chlorambucil, 5-hydroxytryptophan, L-theanine, biotin, levodopa and carbocysteine.

17. The coordination composition of claim 1, wherein the central atoms or ions comprise one or more of Zn, Cu, Mn and Mg, or one or more of Zn²⁺, Cu²⁺, Mn²⁺ and Mg²⁺; the multitopic ligand comprises one or more of 1,1′-(1,4-butanediyl)bis(imidazole), 1,1′-(1,5-pentanedidyl)bis(imidazole), 1,1′-(1,6-hexanedidyl)bis(imidazole) and 1,1′-(1,8-octanedidyl)bis(imidazole); and the pharmaceutical compound comprises one or more of diclofenac and naproxen.

18. The coordination composition of claim 1, wherein the central atoms or ions comprise Cu or Cu²⁺; the pharmaceutical compound comprises one or more of ibuprofen and diclofenac; and the multitopic ligand comprises one or more of the following:

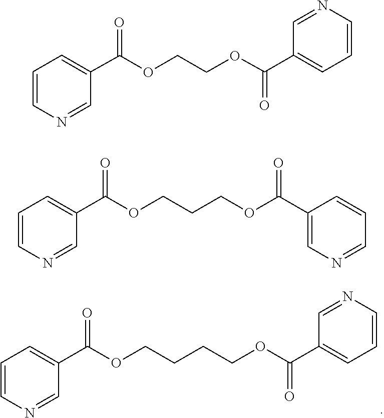

19. The coordination composition of claim 1, wherein the central atoms or ions comprise Cu²⁺; the pharmaceutical compound comprises one or more of ibuprofen and diclofenac, or preferably, ibuprofen; and the multitopic ligand comprises

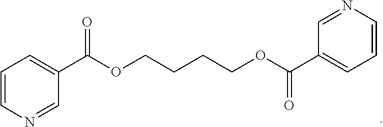 [SMILES: O=C(OCCCCOC(=O)c1cccnc1)c1cccnc1]

20. The coordination composition of claim 1, wherein the coordination composition is selected for forming an amorphous or semi-crystalline solid upon melting followed by cooling, wherein the amorphous or semi-crystalline solid comprises Cu²⁺, ibuprofen, niacin and 1,4-butanediol.

21. The coordination composition of claim 1, wherein the central atoms or ions comprise Zn or Zn²⁺; the pharmaceutical compound comprises one or more of diclofenac, ibuprofen, naproxen and indomethacin; and the multitopic ligand comprises one or more of the following:

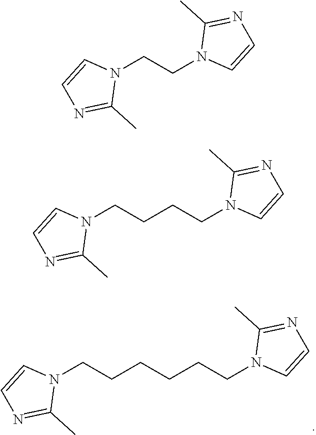 [SMILES: Cc1nccn1CCn1ccnc1C ; Cc1nccn1CCCCn1ccnc1C ; Cc1nccn1CCCCCCn1ccnc1C]

22. The coordination composition of claim 1, wherein the central atoms or ions comprise Zn²⁺; the pharmaceutical compound comprises one or more of diclofenac, ibuprofen, naproxen and indomethacin; and the multitopic ligand comprises

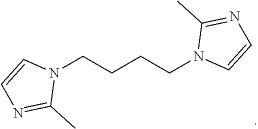 [SMILES: Cc1nccn1CCCCn1ccnc1C]

23. The coordination composition of claim 1, wherein the coordination composition is selected for forming an amorphous or semi-crystalline solid upon melting followed by cooling, wherein the amorphous or semi-crystalline solid is for forming a film or predetermined shape.
